(19) United States
(12) Plant Patent Application Publication
(10) Pub. No.: US 2002/0166151 P1
(43) Pub. Date: Nov. 7, 2002

(54) 'AC- Yamaska' strawberry

(76) Inventor: Shahrokh Khanizadeh, Baie d' Urfe (CA)

Correspondence Address: Therese Otis, P.Ag., Agriculture & Agri-Food, Canada - Horticultural R & D Center, 430 Gouin Blvd, St-Jean-sur-Richelieu, J3B 3E6, CA

(21) Appl. No.: 09/849,818

(22) Filed: May 7, 2001

(51) Int. Cl. A01H 5/00

(52) U.S. Cl. PLT/208

(57) ABSTRACT

A Nordic-type strawberry variety named ‘AC-Yamaska’ combines the characteristics of adaptability to heavy soil conditions, and resistance to low winter temperature (−30° C.), to herbicide terbacil and to leaf and root diseases as well as its high yield of large, dark-red, glossy fruits, and its late ripening period which extends the strawberry harvest.

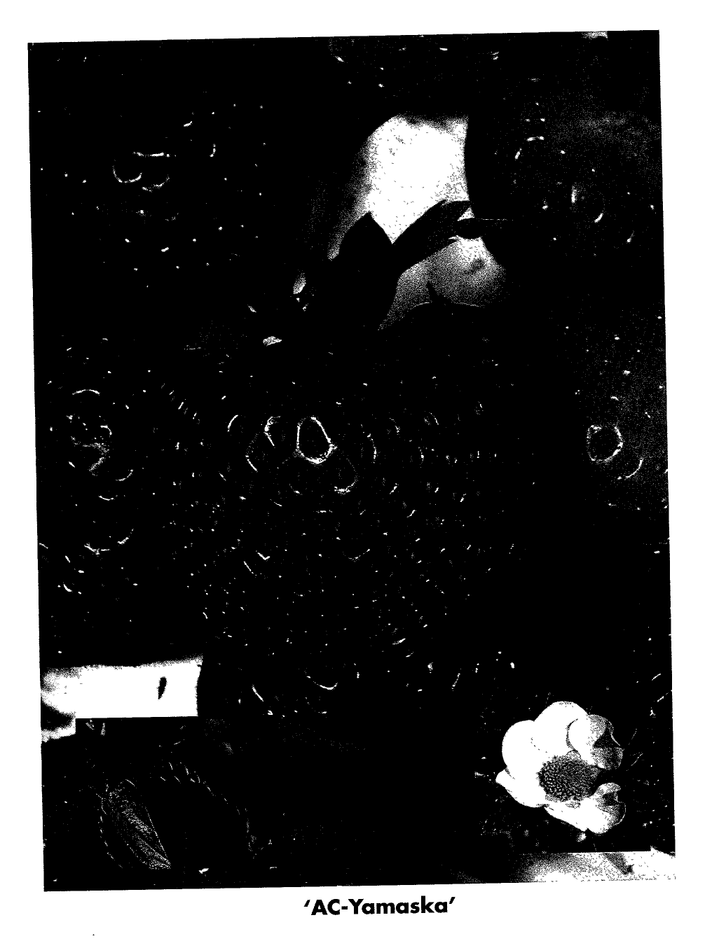

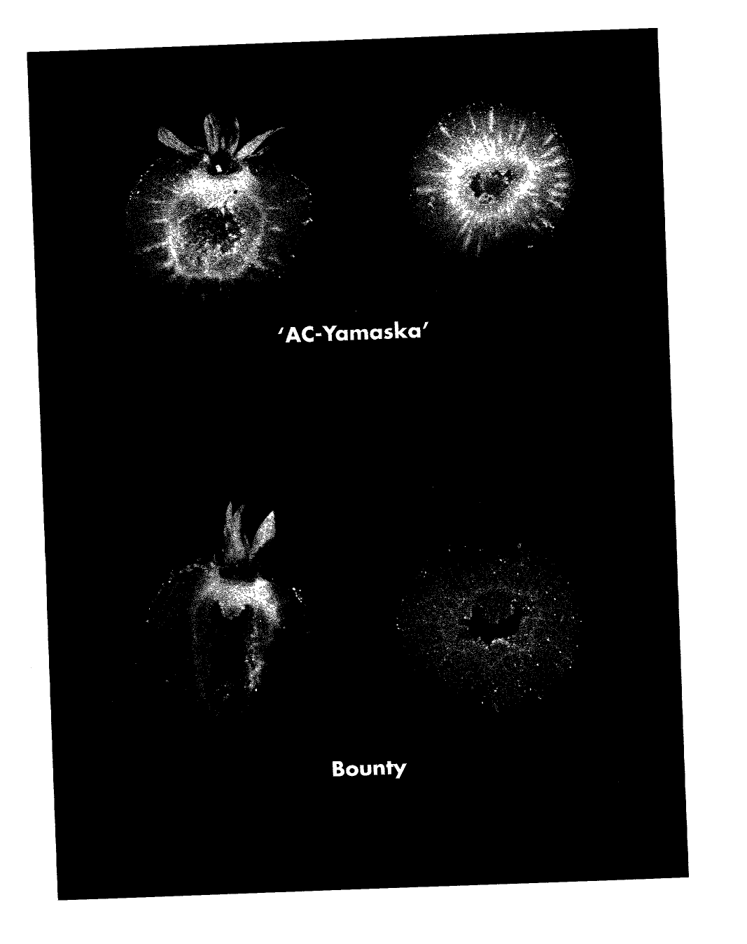

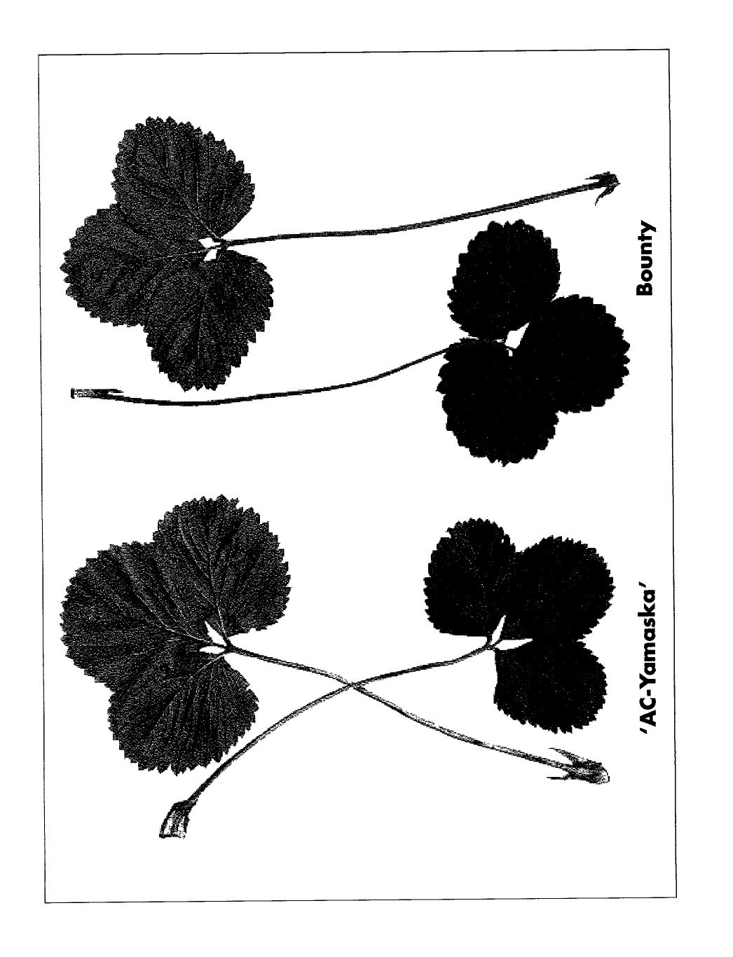

### CROSS REFERENCE TO RELATED APPLICATIONS

None

### STATEMENT REGARDING FED SPONSORED R&D

None

### LATIN NAME OF GENUS AND SPECIES

Fragaria (×ananassa Duch.)

### VARIETY DENOMINATION

‘AC-Yamaska’

### BACKGROUND OF THE INVENTION

The invention relates to a new and distinctive cultivar of strawberry plant named ‘AC-Yamaska’. This cultivar belongs to the genus Fragaria (×ananassa Dutch.). It is a new June-bearing strawberry cultivar bred for Eastern Central Canada and more specifically for Quebec growing conditions. ‘AC-Yamaska’ was released because of its very large, dark red, glossy fruit, and its late ripening period which extends the strawberry harvest.

The plants are moderately resistant to leaf diseases and partially resistant to the principal eastern races of red stele (Phytophthora fragariae Hickman).

### ORGIN OF THE VARIETY

The new cultivar ‘AC-Yamaska’ was tested as SJ89700-1. It is a progeny resulting from a cross made in 1989 by Shahrokh Khanizadeh between two late season cultivars, ‘Pandora’ and ‘Bogota. ‘Pandora’ was originally tested as ‘Jilla 33’ and released by the Horticultural Research Institute in East Malling, UK in 1989. It was used as a parent because of its moderate resistance to verticillium wilt (Verticillium albo-atrum Reinke & Berth.), powdery mildew (Sphaerotheca macularis Wallr. ex Fr.) and grey mold (Botrytis cinerea Pers. ex Fr.). ‘Bogota’ was released in 1978 by the Institute for Horticulture, Plant Breeding, in Wageningen, The Netherlands. It was used as a parent because it produces orange-red fruit late in the season and is moderately resistant to red stele (Phytophthora fragariae Hickman), verticillium wilt and powdery mildew.

‘AC-Yamaska’ has been asexually reproduced by runners and tested at the Agriculture and Agri-Food Canada substation in L'Acadie, Quebec since 1990, and at the Macdonald Campus of McGill University in Ste-Anne-de-Bellevue, during the 1992-1994 fruiting seasons. It was also evaluated from 1996-1998 in controlled semi-commercial sites by our private partners in the province of Quebec, Canada, Lareault Inc., Les Fraises de l'lle d'Orleans Inc. and the Quebec Regional Small Fruit Trials of the Conseil des productions vegetales du Quebec (Provincial Agriculture Department Extension Office).

### DESCRIPTION OF THE FIGURES

1—Photo of fruits (2 sets).

2—Photo of sections of fruit compared to ‘Bounty’ cultivar.

3—Photo of leaves (top and underside) compared to leaves of ‘Bounty’ cultivar.

### DESCRIPTION OF THE VARIETY

‘AC-Yamaska’ is a new June-bearing strawberry cultivar (Fragaria×ananassa Duch.) bred for Eastern Central Canada and more specifically for Quebec growing conditions. ‘AC-Yamaska’ was released because of its very large, dark red, glossy fruit, and its late ripening period which extends the strawberry harvest.

The prefix ‘AC’ stands for ‘Agriculture and Agri-Food Canada’ and ‘Yamaska’ is a town located near the shore of Lake Saint-Peter, a widening of the St. Lawrence River between Richelieu and Nicolet counties in the Province of Quebec. Yamaska is surrounded by rich agricultural lands where farming and market gardening are the main occupations. The word ‘Yamaska’ is derived from a native Indian word meaning “where there is grass under the water” referring to the extensive marshes in the area.

### DETAILED BOTANICAL DESCRIPTION

Plants of ‘AC-Yamaska’ have a medium density, are vigorous and have 2-5 inflorescences per crown. They can tolerate winter temperatures below −30° C. (with 10 cm straw mulch cover). The plants are globose in habit and of medium density. Petioles, which are of sparse pubescence and the hairs of which are upwards, are 11-21 cm long; the leaflets to the amount of three are almost as long as broad, large, dark green, flat, shiny, and obovate, each with about 22-28 serrations. The anthocyanin coloration of the stipules is medium-strong. The leaves of ‘AC-Yamaska’ are dark green in color; they are flat to slightly concave and weakly blistered. Inflorescence are positioned at foliage level with 10 to 12 flowers of medium size (flower petal length: 8.55 mm; petal width: 7.75 mm). The calyx diameter is larger than the corolla; the petals are broader than long and touching to overlapping. ‘AC-Yamaska’ produces a few to a medium number of stolons, which have medium thickness, pubescence and anthocyanin coloration.

‘AC-Yamaska’ has semi-erect fruiting truss of medium length. ‘AC-Yamaska’ produces attractive very large, dark red and shiny fruit that ripen 5 days later than Bounty. Fruit shape is oblate to globose and occasionally short-wedge. There is a very slight difference in shape between primary and secondary fruits. The flesh is dark throughout and fairly firm. The fruit of even color do not show unevenness of the surface with very narrow band without achenes. Achenes stand almost at surface level. The reflexed calyx is smaller than the fruit, levels almost with fruit and has medium adherence to fruit. The flesh of the fruit is even medium red; it is medium sweet and firm with a fine texture and medium acidity. Fresh fruit store well for up to 5 days at room temperature and frozen fruit retain their integrity after thawing.

### PERFORMANCE AND DISEASE RESISTANCE

Yields of ‘AC-Yamaska’ over a four-year period are presented in Table 1 which shows that ‘AC-Yamaska’ had yields similar to those of the cultivars ‘Joliette’, ‘Oka’, ‘Kent’, ‘Glooscap’, ‘Chambly’, and ‘Bounty’. It out-yielded ‘Sparkle’ and ‘Blomidon’. ‘AC-Yamaska’ fruit ripening started 5 days after ‘Bounty’ in our area, but it reached its midpoint earlier than ‘Bounty’ due to its concentrated cropping (short season), therefore it is considered a late cultivar. No symptoms of powdery mildew were noted on ‘AC-Yamaska’ plants since observations began in 1990. ‘AC-Yamaska’ plants were similar to ‘Sparkle’ in terms of susceptibility to the six North American Eastern (NAE) races of Phytophthora fragariae (A1 to A6) based on the presence of red-stele and oospores in the root segments. ‘AC-Yamaska’ had the same level of tolerance to leaf scorch (Diplocarpon earlina Ell. & Ev.) and leaf blight (Dendrophoma obscurans Ell. & Ev.) as ‘Chambly’ and the same susceptibility to leaf spot (Mycosphaerella fragariae (Tul.) Lindau) as ‘Kent’. No signs of gray mold fruit rot (Botrytis cinerea Pers. ex Fr.) were observed on any cultivars except ‘Glooscap’ and ‘Blomidon’ which showed intermediate susceptibility to this disease. The plants are moderately resistant to the herbicide terbacil (SINBAR) (5-chloro-3-(1,1-dimethylethyl)-6-methyl-2,4-(1H,3H )-pyrimidine-dione).

### TEST AND TRAILS

‘AC-Yamaska’ has been tested extensively at Agriculture and Agri-Food Canada, Horticultural R&D Centre substation located at L'Acadie, QC, Canada since 1990. The L'Acadie substation is located 35 km southeast of Montreal Island (lat. 45° N and 46 m elevation). Climate is characterized by extreme low temperatures in winter (<−25° C.); cool, wet, humid conditions in spring; and warm, dry, humid conditions (25° C. to 35° C., 70% RH) in summer. It has a clay loam soil with moderate to low drainage and little snow cover during the winter. ‘AC-Yamaska’ has also been tested at McGill University, Ste-Anne de Bellevue (1992 to 1994) located at the far west of Montreal Island, QC (lat. 45° N and 27 m elevation) close to St. Louis Lake with a sandy clay soil, moderate drainage, and good snow cover; at Ministére de l'Agriculture des Pêcheries et de l'Alimentation du Queébec (MAPAQ), Sainte-Foy, QC (1996 to 1998) located in the west of Quebec city the capital of Quebec province (lat. 46° N and 90 m elevation) with sandy clay loam soil and extreme low temperature in winter; at lies d'Orléans, QC located east of Quebec city the capital of Quebec province (lat. 46° N and 90 m elevation) with sandy clay loam soil and extreme low temperature in winter;; and at Lavaltrie, Quebec located 35 km north of Montreal Island, QC (lat. 45° N and 20 m elevation) with sandy soil and a continental climate. It is also tested in Europe (England and Germany).

### ADAPTATION AND USES

‘AC-Yamaska’ is recommended for Eastern Central Canada, especially in areas where the climate is similar to that in the strawberry production areas of Quebec. It is useful where there is a need to extend the strawberry season. ‘AC-Yamaska’ plants perform very well in sandy soil and they are also adapted to growing in compact or heavy soil. This genotype is presently being evaluated in other provinces of Canada, in the United States, and in Europe.

We claim:

1. A new and distinct cultivar of Fragaria×ananassa Duchh. plant named ‘AC-Yamaska’, as herein shown and described, characterized particularly as to uniqueness by the combined characteristics of adaptability to heavy soil conditions, and resistance to low winter temperature (<−30° C), to herbicide terbacil and to leaf and root diseases as well as its high yield of large, dark-red, glossy fruits, and its late ripening period which extends the strawberry harvest.
